(19) United States
(12) Patent Application Publication
(10) Pub. No.: US 2015/0202045 A1
(43) Pub. Date: Jul. 23, 2015

(54) Bone Implant Apparatus and Method

(71) Applicants: John S. Early, Dallas, TX (US); Andrew S. Kaplan, Burlington, VT (US)

(72) Inventors: John S. Early, Dallas, TX (US); Andrew S. Kaplan, Burlington, VT (US)

(73) Assignee: Bespa, Inc

(21) Appl. No.: 14/162,347

(22) Filed: Jan. 23, 2014

(51) Int. Cl. A61F 2/28

(52) U.S. Cl. CPC A61F 2/28

(57) ABSTRACT

A bone implant apparatus and method includes a programmable device for creating a bone implant. A computer program is connected with the programmable device and the computer program includes a repository of implant programs for standard bones and spacers of preselected dimensions and forms and where the repository of implant programs are customizable and where the bone implant includes a fixation point.

### FIELD OF THE INVENTION

This invention relates to a bone implant apparatus and method. In particular, in accordance with one embodiment, the invention relates to a bone implant apparatus consisting of a programmable device for creating a bone implant. A computer program is connected with the programmable device and the computer program includes a repository of implant programs for standard bones and spacers of preselected dimensions and forms and where the repository of implant programs are customizable and where the bone implant includes a fixation point.

### BACKGROUND OF THE INVENTION

A problem exists with regard to the creation of bone implants and spacers. There are over two hundred bones in the body all of which can be injured or destroyed by trauma, arthritis, tumor, or infection. There are at present, however, only replacements for a fraction of these bones and joints and not for any of the middle segments of the bone in part due to greater difficulty in replicating the shapes of these bones and because of their less frequent involvement. Prior art manufacturers may not have found it profitable enough to invest in treating the diseases of these bones, yet many people suffer from pain in these joints.

Further, the present process to fill bone voids is by a cumbersome and poorly customized hand fashioning or molding for example of cement. Other implants such as bone graft or synthetic bone substitutes are available often as blocks that have to be painstakingly chiseled or carved to shape by the doctor in the operating room. All this takes precious time in the operating room while the patient is anesthetized, possibly losing blood, and often exsanguinated at the limb by a tourniquet—tourniquets to control blood loss may only be used about the limb for a limited time without causing nerve, vessel and tissue damage.

Obviously, time taken to mold and chisel and burr the presently available implantable forms adds time and thus risk and expense to surgery. Furthermore whole bone shapes are only available from organ donors at the present time and available sizes vary significantly and they sometimes do not fit appropriately with the patient undergoing surgery and run the risk of carrying diseases such as HIV and Hepatitis.

In sum, known problems with the prior art bone implants and spacers or space fillers are that the prior art process adds operating room time and thus risk to the patient and the prior art limited range of options significantly limits the exact replication of native bone shapes and cannot meet specifications produced by reproducible methods.

Further problems exist as well. Allografts and spacers presently available have no built-in means for stability and fixation and this is a significant issue when dealing with the structural bony skeleton. The prior art implants and spacers are often squeezed or packed into place or sometimes surgeons can take time and make drill holes for screws and the like through materials while in the operating room if the piece is not too brittle.

Additionally, the prior art does not include implants or spacers that are conformed to become an organic part of the body. For the most part, sterile implants are the norm in order to reduce the risk of infection and rejection.

Thus, there is a need in the art for a bone implant means and method for providing bone implants of any size, shape and form and for all desired sizes, shapes and forms for use in rehabilitative surgery. There is a further need for bone implants that include preselected and propositioned fixation points and when necessary are conformed to bioactively integrate with the implant recipient.

It therefore is an object of this invention to provide a bone implant apparatus and method that enables the creation of bone implants of any and all desired shapes, forms and sizes. Further, it is an object of this invention that data for the creation of preselected bone implant forms, sizes and shapes be contained on a computer program such that the desired bone implant is selectable from a repository of bone implant shapes, forms and sizes as needed and on demand. It is a further object of the invention that the repository be customizable such that existing forms may be conformed as required by particular circumstances and still further that bone implants may be created in response to new data as received. It is a still further object of the invention that bone implants include as desired or required a bio active surface.

### SUMMARY OF THE INVENTION

Accordingly, the bone implant apparatus and method of the present invention, according to one embodiment, includes a programmable device for creating a bone implant. A computer program is connected with the programmable device and the computer program includes a repository of implant programs for standard bones and spacers of preselected dimensions and forms and the repository of implant programs are customizable and where the bone implant includes a fixation point.

All terms used herein are given their common meaning. Thus, a “programmable device” is any machine that is manipulable by computer programming. 3D machines are known in the art that are programmed as needed to create desired parts, for example. Computer programs are contained on computer readable medium and are loaded on and thereby “connected” with machines and devices as is known. “Customizable” is used to describe a computer program that may be modified by the user to effect desired alternative results. “Fixation point” describes a location on the bone implant what is used to secure the bone implant in a desired location.

According to another aspect of the invention, the bone implant consists of structure with a bio-active surface. Here, “bio-active” describes a surface that is biologically active, alive, as will be described more fully hereafter.

In one aspect, the bone implant is a sterilizable spacer. I another aspect, the bone implant is an antibiotic spacer. In a further aspect, the bone implant is made from material FDA approved for implantation. In one aspect, the bone implant is a whole bone implant.

In another aspect, the customizable computer program is customized by comparing a selected implant program for a particular standard bone or spacer with an actual bone and conforming the selected implant program to match selected elements of the actual bone.

In a further aspect, the customizable computer program receives data for a particular bone or spacer and directs the programmable device to create a bone implant or spacer to match that particular bone or spacer without reference to the repository of implant programs for standard bones and implants.

In one aspect, the type of fixation point is selected from a group consisting of: holes, tabs, dowels, flanges and notches.

According to another embodiment of the invention, a bone implant apparatus consists of. a programmable device for creating a bone implant where the bone implant is a 3D bone implant made of FDA approved material. A computer program is connected with the programmable device where the computer program includes a repository of implant programs for standard bones and spacers of preselected dimensions and forms and where the implant programs are customizable wherein the customizable computer program is customized by comparing a selected implant program for a particular standard bone or spacer with an actual bone and conforming the selected implant program to match selected elements of the actual bone and where the bone implant includes at least one fixation point.

According to another aspect, the bone implant consists of structure with a bio-active surface on the exterior or on the interior of the structure. In another aspect, the bone implant is a sterilizable spacer. In a further aspect, the bone implant is an antibiotic spacer. In one aspect, the bone implant is a whole bone implant.

In a further aspect, the customizable computer program receives data for a particular bone or spacer and directs the programmable device to create a bone implant or spacer to match that particular bone or spacer without reference to the repository of implant programs for standard bones and implants.

In another aspect, the fixation point is selected from a group consisting of: holes, tabs, dowels, flanges and notches.

According to another embodiment of the invention, a method of creating a bone implant consists of the steps of:

a. providing a programmable device for creating a bone implant, where the bone implant includes a fixation point; a computer program connected with the programmable device where the computer program includes a repository of implant programs for standard bones and spacers of preselected dimensions and forms and where the implant programs are customizable; and then utilizing the programmable device to create a bone implant by means of the computer program.

In another aspect, the customizable computer program is customized by comparing a selected implant program for a particular standard bone or spacer with an actual bone and conforming the selected implant program to match selected elements of the actual bone.

In a further aspect, the customizable computer program receives data for a particular bone or spacer and directs the programmable device to create a bone implant or spacer to match that particular bone or spacer without reference to the repository of implant programs for standard bones and implants.

In one aspect, the fixation point is selected from a group consisting of: holes, tabs, dowels, flanges and notches.

### DESCRIPTION OF THE DRAWINGS

Other objects, features and advantages of the present invention will become more fully apparent from the following detailed description of the preferred embodiment, the appended claims and the accompanying drawings in which:

FIG. 1 is a schematic of the programmable device of the bone implant apparatus of the present invention according to one embodiment;

FIG. 2 is a side view of a bone implant of the present invention according to one embodiment;

FIG. 3 is a side view of the bone implant of FIG. 2 shown in place in natural bone;

FIG. 4 is a perspective view of a fixation point in the form of a dowel;

FIG. 5 is a side sectional view of a bone implant with a fixation point in the form of a flange with a bio-active surface on the outside;

FIG. 6 is a side sectional view of a bone implant with a fixation point in the form of a notch with a bio-active surface on the hollow inside; and

FIG. 7 is a side sectional view of a bone implant with a nail hole from end to end.

### DETAILED DESCRIPTION OF THE INVENTION

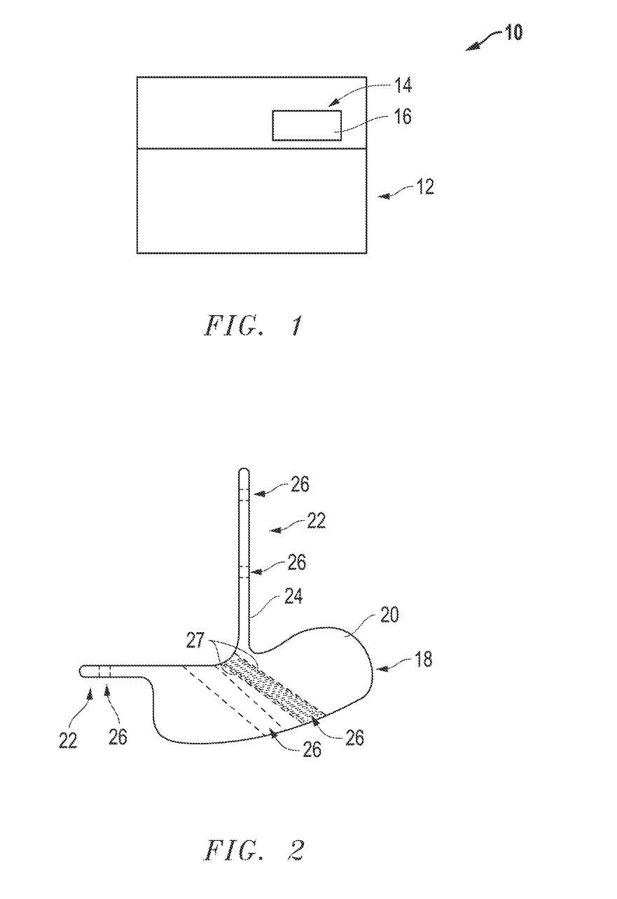

The preferred embodiment of the present invention is illustrated by way of example in FIGS. 1-7. With specific reference to FIGS. 1 and 2, bone implant apparatus 10 according to a preferred embodiment includes a programmable device 12. Programmable device 12 includes computer program 14. Computer program 14 is contained on computer readable medium and “loaded” onto programmable device 12 where it is accessed for use as is known in the art and not described more fully hereafter. Computer program 14 includes a repository of implant programs 16. Implant programs 16 consist of instructions for the creation of forms for standard bones and spacers of preselected dimensions. Implant programs 16 in a preferred embodiment consist of pre-prepared instructions of any and all forms of standard bones and spacers, separately selectable by a user by means of programmable device 12. As with known computer programs and devices, computer program 14 is accessible through programmable device 12 such that computer program 14 may be added to or deleted from as desired. Further, according to one embodiment, any particular implant program 16 may be selected and modified to reflect changes required so as to result in an implant that perfectly fits the parameters of a particular surgical situation.

Additionally, according to one embodiment, programmable device 12 receives data input in the form of a particular needed implant but an implant for which no data is maintained in the repository of implants 16. In this case, the design for the new implant is created directly from the newly received data by the computer program 14. Thereafter, the new implant design may be saved and stored in the repository of implant programs 16. In these ways, computer program 12 is “customizable” as the term is used herein.

Referring, now to FIG. 2, bone implant 18 is shown in a preferred embodiment in the form of a standard bone 20. That is, “standard bone” 20 is an anatomically correct bone shape except for the fixation points 22. FIG. 2 illustrates fixation point 22 in two forms, flanges 24 and through holes 26. As shown, through holes 26 may be located as desired, either through the standard bone 20 or the flange 24 or both as shown. Through hole 26 may be used to thread a wire (not shown) or a screw (not shown) in cooperation with screw threads 27, when desired, or some other device for securing bone implant 18 in place.

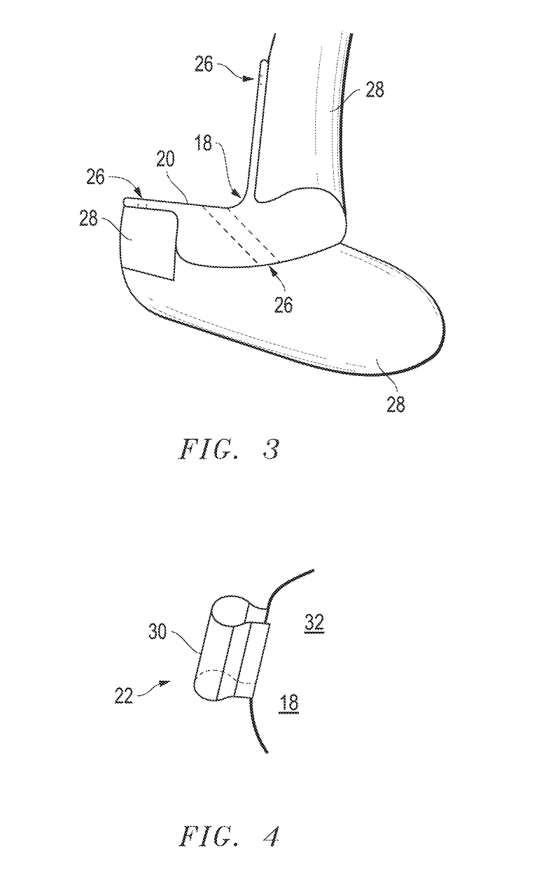

Referring now to FIG. 3, the bone implant 18 of FIG. 2 is shown in place between two sections of natural bone 28. Again, in this embodiment, bone implant 18 is created by programmable device 12 in the form of a standard bone 20. That is, it is anatomically accurate except for fixation points 22, flanges 24. Flanges 24 extend along natural bones 28 and provide a connective surface to assist in fixing bone implant 18 in place. Through holes 26 allow screws (not shown), for example only and not by way of limitation, to pass through flanges 24 so as to further secure bone implant 18 with the natural bone 28.

Referring now to FIGS. 4, 5, 6, and 7 other embodiments of the invention concerning examples of fixation points 22 are illustrated. FIG. 4 illustrates a fixation point 22 in the form of a dowel 30. Dowel 30 is connected with a bone implant 18 which may be a standard bone implant 20 or a spacer 32. Spacer 32 is used in surgical situations to maintain a required spacing. Thus, spacer 32 does not necessarily need to be anatomically correct. In any event, dowel 30 is connected to the bone implant by any means now known or hereafter developed or is created as an integral part of the bone implant 18.

FIG. 5 illustrates a spacer 32 with a flange 24 or shoulder used to aid in fixing the spacer 32 in place and FIG. 6 shows a fixation point 22 in the form of a notch 34 or ledge used for the same purpose. It may be that flange 24 and notch 34 cooperate as male-female parts to join two separate spacers 32 together.

Still referring to FIGS. 5 and 6, another preferred embodiment of the invention is illustrated. FIG. 5 illustrates bio-active surface 36 on the outside surface 38 of spacer 32. Certainly, bio-active surface 36 may be located as desired on standard bone implants 20 as well. In whatever form, the shapes maybe the first layer upon which another layer forms an outer enamel that is a biologically active surface 36. This could be a porous layer for ingrowth of cartilage cells or stem cells. Conversely it, as illustrated in FIG. 6, bio-active surface 36 could be an incubator layer where cells are able to migrate, cultivate and grow from stem cell line or cartilage cells in order to cover parts of the inner core 40 with a bioactive outer surface layer 36. Certainly, the ends of implants 18 could be a bioactive layer 36 that features cartilage cells and the environment to grow and sustain them. These ends could create parts that act as joints, for example only and not by way of limitation.

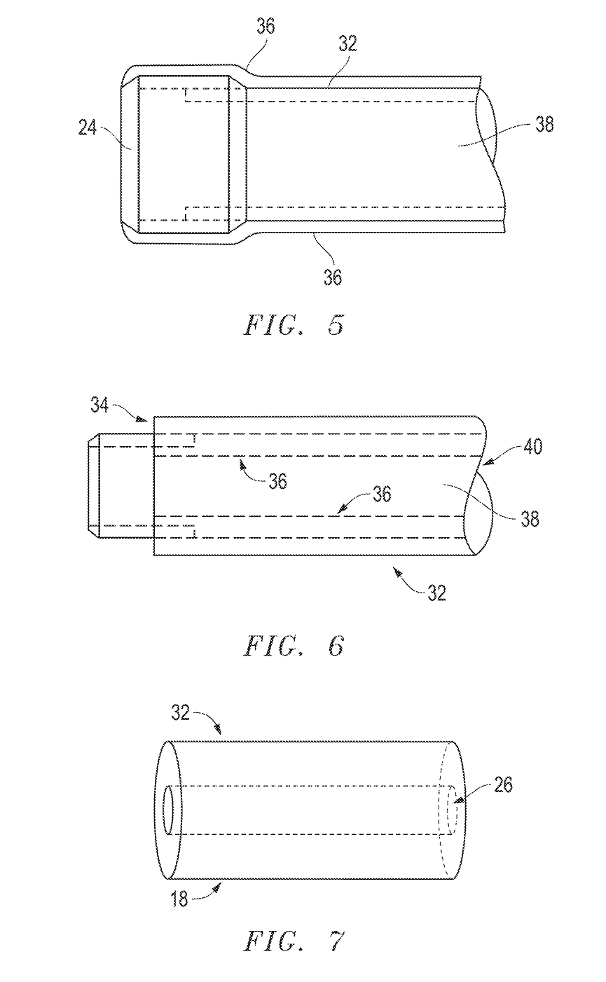

FIG. 7 illustrates a bone implant 18 and or spacer 32, created by bone implant apparatus and method 10 to include a precise through hole 26 conformed to compatible with various diameters of intramedullary nails, known in the art and not shown, that can be placed down the through hole 26 for securing the bone implant 18 and/or spacer 32 in position.

By way of further explanation, the bone implant apparatus and method 10 of the present invention creates a bank or portfolio of exact replica designed sterilizable, implantable bone implants 18, either standard bone shapes 20 or bone spacers 32, for missing or destroyed bone parts by using a programmable device 10, such as found in 3-D printing technology or other technology, to assemble the portfolio from which a bone implant 18 specifically requested by a healthcare provider can then be sent directly to the medical provider or institution or can be sent via data file from the portfolio to their 3-D printer on site. Additionally, the present invention provides for the ability to create custom 3-D design implants from, for example only and not by way of limitation, a CT scan image of the contralateral intact bone in the patient to be used on the opposite side, which has been damaged. It is understood that printing materials can be of biologic or non-biologic material that have already been approved by the FDA for shaping and then implanting. Traditionally, this has been by hand molding in the operating room but has never been printed or kept in stock as a skeletal bank or portfolio of options from which tertiary care centers can order or choose.

The present invention includes a repository of implant programs for the creation of three different types of bone implants 18:
1) Synthetic bone shapes of standard bones 20;
2) Implantable spacers 32 that may be sterilizable or include the ability to release antibiotics into the surrounding tissue, for example only and not by way of limitation; and
3) Structures with biologically active surfaces 36 made from patients cultured stem cells or cartilage cells coated as a surface layer over the shaped substrate layer.

Anticipated bone implants 18 created by the bone implant apparatus 10 include, for example only:
1) Bone implants 18 ordered from in stock size and shape;
2) Using CT scan images created by a surgeon and requested for custom manufacturing at the surgeon's request. In this manner computer program 14 is customizable such that bone implant 18 designs may be based from the CT scan that shows the defect size in relation to or compared to the contralateral normal side to then determine what specific shapes must be created.
3) Bone implants 18 created with layered structures created from patients' cell cultures.
4) Bone implant 18 structures printed with pre-programmed or generic fixation points 22 in many forms upon request. Fixation points 22 impart greater stability to the bone implants 18, standard bone 20 or spacers 32, when placed in the defect. Because the computer program 14 is customizable, physicians may request predrilled screw holes 26 or pin channels that can be preprogrammed into the bone implant 18 design so as to allow fixation to adjacent bone Likewise fixation of long bones may be pre-program with internal or external flanges 24 or tabs that serve to attach it to adjacent bony segments to enhance stability or ingrowth or bones may have central canal internal diameters programmed in to allow rod placement through them centrally to then be able to connect them to other bones or parts of bone segments, for example only.

By means of the bone implant apparatus and method 10 of the present invention, surgeons may pre-manufacture or reproduce any needed bone shape or bone defect filler and can have a bank of standard size shapes for whole bones pre-made and available to send along or as a data file to be made at the recipient site. This is a major change from the prior technology as it enables surgeons to avoid the problems associated with the present requirements to shape and carve implant fillers in the operating room.

By way of further explanation and contrast, allografts and spacers presently available have no predefined fixation points 22 or channels to allow fixation of bone implants 18 to native bone. Presently bone implants 18 are often squeezed or packed into place or sometimes surgeons can take time and make drill holes through materials while in the operating room if the piece is not too brittle. But none of the present materials have built-in means for stability and fixation and this is a significant issue when dealing with the structural bony skeleton. Applicants' present invention creates implantable bone implants 18 that have ready fixation points 22. Multiple options for fixation maybe preprogrammed into the bone implants 18 of the present invention at the design phase in programmable device 12 in stock components, or custom options by surgeon requests could be premolded in for specific screw passage, or wire or rod passage or even end point fixation by fixation tabs or flanges that attach the bone implant 18 to the adjacent natural bone 28 structures. These premolded implants then could be almost instantaneously “snapped into place” and secured by fixation in place through passages that were already preset in proper direction with proper threading etc. for securing with metal hardware. This represents a major change from prior available technology that had no easy or preprogrammed means of fixation whatsoever.

The present invention creates a bone implant 18 immediately useable, implantable (because it is the correct size and shape) and stable (because it can be easily fixated in place with screws or wires, etc . . . )—something severely lacking with the present technology.

Applicants intend that the bone implants 18 of the present invention use any of the already FDA approved orthopedic use materials for implant to create the basic whole bone or bone defect shapes. This includes biologic and synthetic materials such as bone, cartilage cells, stem cells, dermal cells, hydroxy-appetite, calcium phosphate, methylmethacrylate cement, Implantable plastics and metals.

Secondly a bio active structural combination of materials may be created: The basic bone shapes maybe the first layer upon which another layer forms an outer enamel that is a. biologically active layer. This could be a porous layer for ingrowth of cartilage cells or stem cells. Conversely in could be an incubator layer where cells are able to migrate, cultivate and grow from stem cell line or cartilage cells in order to cover parts of the Inner core with a bio-active outer surface layer. In this second layer, the ends of implants could be a bio-active layer that features cartilage cells and the environment to grow and sustain them. These ends could create parts that act as joints.

In sum, it is proposed to create a bank of bone shapes through the programmable device 10, 3-D printing process or otherwise, from various materials for implanting into the body that can be sterilizable prior to implantation. The bone implants 18, either standard bone 20 or spacers 32, may be manufactured and shipped directly to doctors and hospitals in need of the implants or conversely printing speculations could be placed in a data file which could then be sent to healthcare operating rooms or institutions for printing of the implant on site if they had their own capable 3-D printer/programmable device 10. Materials to be used could be moldable bone chips with a hardening agent to hold the shapes together or hydroxyapatite or methylmethacrylate bone cement or any of the bio composite materials such as calcium phosphate powders that are available for orthopedic implantation. Bone implants 18 could be used as sterile spacers when bone has been destroyed or occasionally when a bone has been extruded from the body from trauma and is missing or when a bone must be surgically removed from the body because of infection or tumor or destruction i.e. when it has been pulverized. Spacers 32 could be used in a temporary manner as an internal splint possibly sometimes replacing the need for external fixation devices, which have added risks of infection and add trauma to adjacent bones and soft tissue structures. Sometimes spacers 32 could be left in place for more extended periods or occasionally on a permanent basis depending on conditions of the wound medical conditions of the patient, life expectancy of the patient, the desires of the patient and physician based on the response to the spacer. Custom antibiotic spacers 32 could be created and used to maintain soft tissue tension after infected implants were removed after a failed total joint replacement where the spacer 32 could be printed from exact specs of the previous metal implant which had to come out. Bone spacers or partial bone spacers could conceivably be used to treat arthritic joints destroyed on the articular side of the bone by the arthritic process. Anatomically printed spacers 32 present a smooth congruous anatomic surface at the joint level once they were fixed into place. There could be a multilayered spacer with a structural core and a bio-active cellular layer 36 on the outer enamel.

Again, while some spacers 32 will wedge neatly or fit snugly into place between bony segments without need for further fixation, some may be further fixated by being preprogrammed within programmable device 12 with various fixation augmentations and applications such as:
1) Pre-threaded holes for lag or locking screw placement;
2) Built in channels for pin passage;
3) Printed tabs, dowels, flanges, notches or any combination of such at the end of bone implant 18 so as to capture, fixate into or interdigitate with adjacent natural bone 28, thereby keeping implant structures securely in place and controlling against forces of rotation, angulation, longitudinal collapse, or extrusion.
   a. Tabs project out of the manufactured component onto the interface surface of the native bone. They are used when the anatomy will accommodate some distraction when placing the manufactured segment. Using customizable computer program 14, a template will be manufactured based on the native anatomy and provided with the component to ensure accurate placement of the receiving hole or holes.
   b. Dowels 30 would be another available fixation method where a projection off of the manufactured implant would key into a prepared slot in the native bone. A manufactured guide matching existing anatomy will be produced to assist in proper receptor placement.
   c. Flanges 24 can be placed internally or externally on any manufactured implant to engage the native bone. Fixation holes 26 can also be placed in the flanges 24 to provide points for additional fixation.
4) As well, specific internal diameter sizes can be fabricated within the spacers to allow for passage of an internal fixation nail.
5) Some implants, fixated in place, will serve as a form of “hone plate” or flange 24 by spanning a defected segment.

These fixation portals or augmentations may be preprogrammed and/or custom locations for augmentation may be specifically requested by physician surgeons for specific application. Additionally the outer contours of bone implants 18 may be specifically contoured to accommodate specific physician requests for fixation methods. Each will come with cutting templates to provide a matched mating surface for the replacement bone to the natural bone.

The description of the present embodiments of the invention has been presented for purposes of illustration, but is not intended to be exhaustive or to limit the invention to the form disclosed. Many modifications and variations will be apparent to those of ordinary skill in the art. As such, while the present invention has been disclosed in connection with an embodiment thereof, it should be understood that other embodiments may fall within the spirit and scope of the invention as defined by the following claims.

What is claimed is:

1. A bone implant apparatus comprising:
a. a programmable device for creating a bone implant;
b. a computer program connected with said programmable device wherein said computer program includes a repository of implant programs for standard bones and spacers of preselected dimensions and forms wherein said implant programs are customizable; and
c. wherein said bone implant includes a fixation point.

2. The apparatus of claim 1 wherein said bone implant consists of structure with a bio-active surface.

3. The apparatus of claim 1 wherein said bone implant is a sterilizable spacer.

4. The apparatus of claim 1 wherein said bone implant is an antibiotic spacer.

5. The apparatus of claim 1 wherein said bone implant is made from material FDA approved for implantation.

6. The apparatus of claim 1 wherein said bone implant is a whole bone implant.

7. The apparatus of claim 1 wherein said customizable computer program is customized by comparing a selected implant program for a particular standard bone or spacer with an actual bone and conforming the selected implant program to match selected elements of said actual bone.

8. The apparatus of claim 1 wherein said customizable computer program receives data for a particular bone or spacer and directs said programmable device to create a bone implant or spacer to match that particular bone or spacer without reference to said repository of implant programs for standard bones and implants.

9. The apparatus of claim 1 wherein said fixation point is selected from a group consisting of:
holes, tabs, dowels, flanges and notches.

10. A bone implant apparatus comprising:
a. a programmable device for creating a bone implant wherein said bone implant is a 3D bone implant made of FDA approved material;
b. a computer program connected with said programmable device wherein said computer program includes a repository of implant programs for standard bones and spacers of preselected dimensions and forms wherein said implant programs are customizable wherein said customizable computer program is customized by comparing a selected implant program for a particular standard bone or spacer with an actual bone and conforming the selected implant program to match selected elements of said actual bone; and
c. wherein said bone implant includes at least one fixation point.

11. The apparatus of claim 10 wherein said bone implant consists of structure with a bio-active surface on the exterior or on the interior of said structure.

12. The apparatus of claim 10 wherein said bone implant is a sterilizable spacer.

13. The apparatus of claim 10 wherein said bone implant is an antibiotic spacer.

14. The apparatus of claim 10 wherein said bone implant is a whole bone implant.

15. The apparatus of claim 10 wherein said customizable computer program receives data for a particular bone or spacer and directs said programmable device to create a bone implant or spacer to match that particular bone or spacer without reference to said repository of implant programs for standard bones and implants.

16. The apparatus of claim 10 wherein said fixation point is selected from a group consisting of:
holes, tabs, dowels, flanges and notches.

17. A method of creating a bone implant comprising:
a. providing a programmable device for creating a bone implant, wherein said bone implant includes a fixation point; a computer program connected with said programmable device wherein said computer program includes a repository of implant programs for standard bones and spacers of preselected dimensions and forms wherein said implant programs are customizable; and b. utilizing said programmable device to create a bone implant by means of said computer program.

18. The method of claim 17 wherein said customizable computer program is customized by comparing a selected implant program for a particular standard bone or spacer with an actual bone and conforming the selected implant program to match selected elements of said actual bone.

19. The method of claim 17 wherein said customizable computer program receives data for a particular bone or spacer and directs said programmable device to create a bone implant or spacer to match that particular bone or spacer without reference to said repository of implant programs for standard bones and implants.

20. The method of claim 17 wherein said fixation point is selected from a group consisting of: holes, tabs, dowels, flanges and notches.
